(19) United States
(12) Patent Application Publication
(10) Pub. No.: US 2026/0002900 A1
(43) Pub. Date: Jan. 1, 2026

(54) SENSOR AND METHOD FOR MANUFACTURING THE SAME

(71) Applicant: KABUSHIKI KAISHA TOSHIBA, Tokyo (JP)

(72) Inventors: Ko YAMADA, Yokohama Kanagawa (JP); Yumi HAYASHI, Ayase Kanagawa (JP); Naoki HIRAMATSU, Kawasaki Kanagawa (JP); Hiroshi HAMASAKI, Hiratsuka Kanagawa (JP)

(73) Assignee: KABUSHIKI KAISHA TOSHIBA, Tokyo (JP)

(21) Appl. No.: 19/230,183

(22) Filed: Jun. 6, 2025

(30) Foreign Application Priority Data
Jul. 1, 2024 (JP) 2024-106347

(51) Int. Cl. G01N 27/12; G01N 33/00

(52) U.S. Cl. CPC G01N 27/125; G01N 33/0027

(57) ABSTRACT

According to one embodiment, a sensor includes an element portion. The element portion includes an element layer including a first face, a first electrode, a second electrode, and a first member. The first electrode is provided on the first face. The second electrode is provided on the first face. A direction from the second electrode to the first electrode is along a first direction. The first member includes an oxide. The first face includes a first region and a second region. The first region is recessed with respect to the second region. The first region and the second region overlap at least a part of the first electrode in a third direction. At least a part of the first electrode is between the first region and the first member in the third direction.

### CROSS-REFERENCE TO RELATED APPLICATIONS

This application is based upon and claims the benefit of priority from Japanese Patent Application No. 2024-106347, filed on Jul. 1, 2024; the entire contents of which are incorporated herein by reference.

### FIELD

Embodiments described herein relate generally to a sensor and a method for manufacturing the same.

### BACKGROUND

For example, there is a sensor for detecting a detection target such as gas, etc. It is desirable to improve the characteristics of the sensor.

### BRIEF DESCRIPTION OF THE DRAWINGS

FIG. 1 is a schematic plan view illustrating a sensor according to a first embodiment.

FIG. 2 is a schematic cross-sectional view illustrating the sensor according to the first embodiment;

FIG. 3 is a schematic cross-sectional views illustrating the sensor according to the first embodiment;

FIGS. 4A and 4B are schematic cross-sectional views illustrating a part of the sensor according to the first embodiment;

FIG. 5 is a schematic plan view illustrating a sensor according to the first embodiment;

FIGS. 6A and 6B are schematic cross-sectional views illustrating a sensor according to the first embodiment;

FIG. 7 is a schematic plan view illustrating a sensor according to the first embodiment;

FIG. 8 is a graph illustrating a sensor according to the first embodiment; and

FIGS. 9A to 9C are schematic cross-sectional views illustrating a method for manufacturing a sensor according to the second embodiment.

### DETAILED DESCRIPTION

According to one embodiment, a sensor includes an element portion. The element portion includes an element layer including a first face, a first electrode, a second electrode, and a first member. The first electrode is provided on the first face. The second electrode is provided on the first face. A direction from the second electrode to the first electrode is along a first direction. The first member includes an oxide. The first face includes a first region and a second region. A direction from a second position in a second direction of the second region to a first position in the second direction of the first region is along the second direction. The second direction crosses the first direction. The first region is recessed with respect to the second region. The first region and the second region overlap at least a part of the first electrode in a third direction. The third direction crosses a plane including the first direction and the second direction. At least a part of the first electrode is between the first region and the first member in the third direction.

Various embodiments are described below with reference to the accompanying drawings.

The drawings are schematic and conceptual; and the relationships between the thickness and width of portions, the proportions of sizes among portions, etc., are not necessarily the same as the actual values. The dimensions and proportions may be illustrated differently among drawings, even for identical portions.

In the specification and drawings, components similar to those described previously or illustrated in an antecedent drawing are marked with like reference numerals, and a detailed description is omitted as appropriate.

### First Embodiment

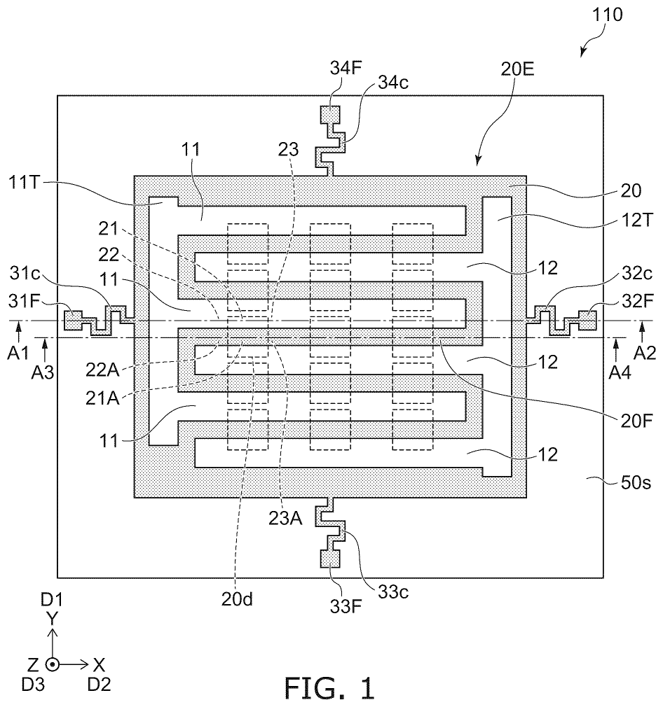

FIG. 1 is a schematic plan view illustrating a sensor according to a first embodiment.

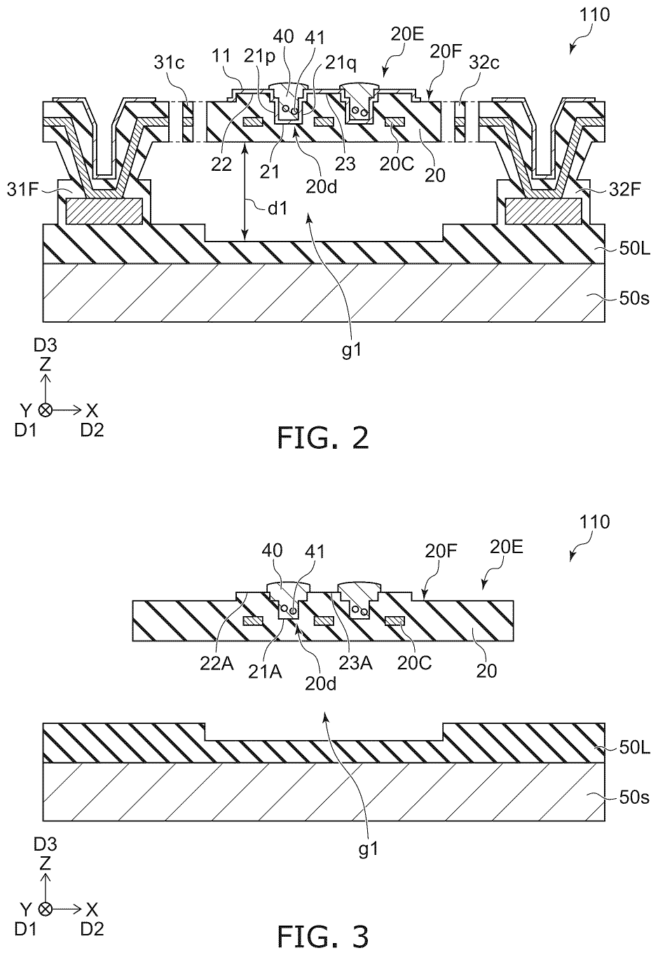

FIGS. 2 and 3 are schematic cross-sectional views illustrating the sensor according to the first embodiment.

FIG. 2 is a cross-sectional view taken along the line A1-A2 in FIG. 1. FIG. 3 is a cross-sectional view taken along the line A3-A4 in FIG. 1.

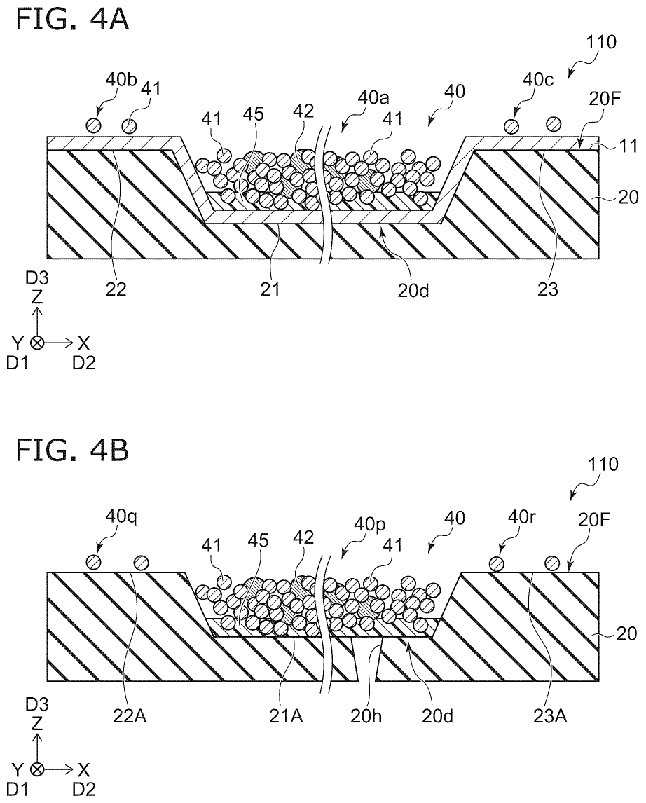

FIGS. 4A and 4B are schematic cross-sectional views illustrating a part of the sensor according to the first embodiment.

As shown in FIGS. 1 to 3, a sensor 110 according to the embodiment includes an element portion 20E.

The element portion 20E includes an element layer 20, a first electrode 11, a second electrode 12, and a first member 40. The element layer 20 includes a first face 20F. The first electrode 11 is provided on the first face 20F. The second electrode 12 is provided on the first face 20F. A direction from the second electrode 12 to the first electrode 11 is along a first direction D1.

The first direction D1 is defined as a Y-axis direction. One direction perpendicular to the Y-axis direction is defined as an X-axis direction. A direction perpendicular to the Y-axis and X-axis directions is defined as a Z-axis direction.

The element layer 20 is along the X-Y plane. The element layer 20 is layered. The element layer 20 may be, for example, a membrane. The element layer 20 is, for example, insulating.

The first member 40 includes an oxide. The first member 40 includes, for example, a plurality of particles (first particles 41) including an oxide (see FIG. 2). At least a part of the first member 40 may be provided on the element layer 20. A part of the first member 40 may be provided on the first electrode 11 and the second electrode 12.

The first face 20F of the element layer 20 includes a first region 21 and a second region 22. A direction from a second position in the second direction D2 of the second region 22 to a first position in the second direction D2 of the first region 21 is along the second direction D2. The second direction D2 crosses the first direction D1. For example, the direction from the second region 22 to the first region 21 crosses the first direction D1. The second direction D2 is, for example, the X-axis direction.

As shown in FIG. 2, the first region 21 is recessed with respect to the second region 22. The first region 21 corresponds to at least a part of the recess (first recess 20d). The second region 22 may correspond to a protrusion.

In this example, the first region 21 and the second region 22 overlap at least a part of the first electrode 11 in a third direction D3. The third direction D3 crosses a plane including the first direction D1 and the second direction D2. The third direction D3 may be, for example, the Z-axis direction.

As shown in FIG. 2, at least a part of the first electrode 11 is located between the first region 21 and the first member 40 in the third direction D3. In FIG. 1, the first member 40 is omitted. In the embodiment, the first electrode 11 is spatially separated from the second electrode 12. The first member 40 may be provided in at least a part of the region between the first electrode 11 and the second electrode 12. The characteristics of the oxide included in the first member 40 change depending on a detection target existing around the element portion 20E. In response to the change in the characteristics of the oxide, the electrical resistance between the first electrode 11 and the second electrode 12 changes depending on the detection target. The detection target can be detected by detecting the change in electrical resistance. The detection target is, for example, a gas or a liquid.

Thus, the first electrode 11 and the second electrode 12 are configured so that the electrical resistance between the first electrode 11 and the second electrode 12 changes depending on the detection target around the element portion 20E. The detection target may include, for example, hydrogen. The sensor 110 may be, for example, a gas sensor.

As described above, the first region 21 is recessed with respect to the second region 22. For example, the recess (first recess 20d) is provided in the first face 20F. Thereby, the plurality of first particles 41 included in the first member 40 become to be stably and uniformly provided in the recess. For example, in a reference example in which a recess is not provided, the plurality of first particles 41 tend to be unevenly present in the element layer 20. In contrast, in the embodiment, the plurality of first particles 41 are stably and uniformly provided in the recess. Thereby, the electrical resistance becomes uniform. The characteristics of the change in electrical resistance in response to changes in the detection target are stabilized. For example, stable and highly accurate detection is possible. According to the embodiment, a sensor capable of obtaining stable characteristics can be provided.

In one example, a liquid including the plurality of first particles 41 may be applied onto the element layer 20 and the electrodes. By removing the solvent in the liquid, the first member 40 including the plurality of first particles 41 can be formed. In this manner, the plurality of first particles 41 in the liquid efficiently and stably gather in the recesses. For example, self-accumulation occurs in the plurality of first particles 41. A sensor with stable characteristics can be provided.

As shown in FIG. 4A, the first member 40 includes the plurality of first particles 41 including an oxide. The oxide includes, for example, oxygen and at least one selected from the group consisting of tin, zinc, tungsten, molybdenum, and indium. The average diameter of the plurality of first particles 41 may be, for example, not less than 10 nm and not more than 500 nms.

As shown in FIG. 4A, the first member 40 may include a plurality of second particles 42. The plurality of second particles 42 may include, for example, at least one selected from the group consisting of platinum, gold, silver, copper, aluminum, and titanium nitride. By providing the plurality of second particles 42, 30 for example, high sensitivity is easily obtained. The plurality of second particles 42 may function, for example, as a catalyst.

As shown in FIG. 4A, the first member 40 may include a resin 45. The resin 45 may be, for example, a polymer.

At least one of the first electrode 11 or the second electrode 12 may include at least one selected from the group consisting of platinum, gold, silver, copper, aluminum, and titanium nitride. For example, the surface part of the first electrode 11 may include at least one selected from the group consisting of platinum, gold, silver, copper, aluminum, and titanium nitride. For example, the surface part of the second electrode 12 may include at least one selected from the group consisting of platinum, gold, silver, copper, aluminum, and titanium nitride. These materials may function as, for example, a catalyst. The region including these materials may be in contact with the first member 40. High sensitivity is easily obtained.

As shown in FIGS. 1 and 2, the first face 20F may further include a third region 23. A first position in the second direction D2 of the first region 21 is located between a second position in the second direction D2 of the second region 22 and a third position in the second direction D2 of the third region 23. The first region 21 is recessed with respect to the third region 23. The third region 23 overlaps a part of the first electrode 11 in the third direction D3. The first region 21 between the second region 22 and the third region 23 corresponds to a part of the first recess 20d.

By providing the recess (first recess 20d), the first member 40 selectively gathers in the recess. The first member 40 may not be provided in a region that is not a recess. For example, the first member 40 may not overlap the second region 22 in the third direction D3. Alternatively, the density of the plurality of first particles 41 in the second region 22 may be lower than the density of the plurality of first particles 41 in the first region 21.

In the embodiment, the height (depth) of the step formed between the first region 21 and the second region 22 may be, for example, not less than 1 μm and not more than 5 μm. The step corresponds to the distance in the third direction D3 between the position of the first region 21 in the third direction D3 and the position of the second region 22 in the third direction D3.

For example, the first member 40 may include a first overlapping portion 40a and a second overlapping portion 40b (see FIG. 4A). The first overlapping portion 40a overlaps the first region 21 in the third direction D3. The second overlapping portion 40b overlaps the second region 22 in the third direction D3. A first amount of the oxide (e.g., the plurality of first particles 41) per unit area in the first overlapping portion 40a is greater than a second amount of the oxide (e.g., the plurality of first particles 41) per unit area in the second overlapping portion 40b. The in-plane distribution of the plurality of first particles 41 is appropriately controlled. High sensitivity is easily and stably obtained.

For example, the first member 40 may include a third overlapping portion 40c (see FIG. 4A). The third overlapping portion 40c overlaps the third region 23 in the third direction D3. The first amount of the oxides (e.g., the plurality of first particles 41) per unit area in the first overlapping portion 40a is greater than a third amount of the oxide (e.g., the plurality of first particles 41) per unit area in the third overlapping portion 40c. The in-plane distribution of the plurality of first particles 41 is appropriately controlled. High sensitivity is easily and stably obtained.

As shown in FIG. 1, the first electrode 11 may extend along the second direction D2. The second electrode 12 may extend along the second direction D2.

As shown in FIG. 2, the first face 20F may include a first side face 21p and a second side face 21q. The first side face 21p is located between the first region 21 and the second region 22. The second side face 21q is located between the first region 21 and the third region 23. The first side face 21p and the second side face 21q cross the second direction D2. For example, a part of the first electrode 11 is located between the first side face 21p and at least a part of the first member 40. For example, another part of the first electrode 11 is located between at least a part of the first member 40 and the second side face 21q. The first side face 21p and the second side face 21q correspond to a part of the side face of the first recess 20d. At least a part of the first member 40 is provided between the two side faces. The plurality of first particles 41 are stably collected in the first recess 20d.

As described below, at least a part of the first side face 21p may be inclined with respect to the third direction D3. At least a part of the second side face 21q may be inclined with respect to the third direction D3.

As shown in FIGS. 1 and 3, the first face 20F may further include a first inter-electrode region 21A. A position of the first inter-electrode region 21A in the first direction D1 is located between a position of the second electrode 12 in the first direction D1 and a position of the first electrode 11 in the first direction D1. The direction from the position of the first inter-electrode region 21A in the first direction D1 to the position of the first region 21 in the first direction D1 is along the first direction D1. The first inter-electrode region 21A is recessed with respect to the second region 22. At least a part of the first inter-electrode region 21A overlaps the first member 40 in the third direction D3. For example, at least a part of the first member 40 (the plurality of first particles 41) is provided on the first inter-electrode region 21A. The first inter-electrode region 21A is a part of the first recess 20d. The first inter-electrode region 21A may be recessed with respect to the first region 21.

As shown in FIG. 1, the first face 20F may include a first inter-electrode region 21A and a second inter-electrode region 22A. The position of the first inter-electrode region 21A in the first direction D1 and the position of the second inter-electrode region 22A in the first direction D1 are located between the position of the second electrode 12 in the first direction D1 and the position of the first electrode 11 in the first direction D1. The direction from the second inter-electrode position of the second inter-electrode region 22A in the second direction D2 to the first inter-electrode position of the first inter-electrode region 21A in the second direction D2 is along the second direction D2.

As shown in FIG. 3, the first inter-electrode region 21A is recessed with respect to the second inter-electrode region 22A. At least a part of the first inter-electrode region 21A overlaps the first member 40 in the third direction D3. The first inter-electrode region 21A corresponds to a part of the first recess 20d. The first member 40 (plurality of first particles 41) gather in the first inter-electrode region 21A. High accuracy detection is stably possible.

In the embodiment, the height (depth) of the step formed between the first inter-electrode region 21A and the second inter-electrode region 22A may be, for example, not less than 1 μm and not more than 5 μm. The step corresponds to the distance in the third direction D3 between the position in the third direction D3 of the first inter-electrode region 21A and the position in the third direction D3 of the second inter-electrode region 22A.

For example, the first member 40 does not overlap the second inter-electrode region 22A in the third direction D3. Alternatively, the density of the first particles 41 in the second inter-electrode region 22A is lower than the density of the first particles 41 in the first inter-electrode region 21A.

As shown in FIG. 4B, for example, the first member 40 includes a first inter-electrode portion 40p and a second inter-electrode portion 40q. The first inter-electrode portion 40p overlaps the first inter-electrode region 21A in the third direction D3. The second inter-electrode portion 40q overlaps the second inter-electrode region 22A in the third direction D3. The amount of the oxide (e.g., the plurality of first particles 41) per unit area in the first inter-electrode portion 40p (first inter-electrode amount) is greater than the amount of the oxide (e.g., the plurality of first particles 41) per unit area in the second inter-electrode portion 40q (second inter-electrode amount). The in-plane distribution of the plurality of first particles 41 is appropriately controlled. High sensitivity is easily and stably obtained.

As shown in FIG. 1, the first face 20F may further include a third inter-electrode region 23A. The position of the first inter-electrode region 21A in the second direction D2 is located between the position of the second inter-electrode region 22A in the second direction D2 and the position of the third inter-electrode region 23A in the second direction D2.

As shown in FIG. 3, the first inter-electrode region 21A is recessed with respect to the third inter-electrode region 23A.

As shown in FIG. 4B, the first member 40 may include a third inter-electrode portion 40r. The third inter-electrode portion 40r overlaps the third inter-electrode region 23A in the third direction D3. The amount of the oxide (e.g., the plurality of first particles 41) per unit area in the first inter-electrode portion 40p (first inter-electrode amount) is greater than the amount of the oxide (e.g., the plurality of first particles 41) per unit area in the third inter-electrode portion 40r (third inter-electrode amount). The in-plane distribution of the plurality of first particles 41 is appropriately controlled. High sensitivity is easily and stably obtained.

As shown in FIG. 4B, the element layer 20 may include a hole 20h. The hole 20h penetrates the element layer 20 along the third direction D3. For example, the hole 20h may be connected to the first inter-electrode region 21A. At least a part of the hole 20h may be considered to be the first inter-electrode region 21A. For example, at least a part of the hole 20h is recessed with respect to the second inter-electrode region 22A. As described below, if a gap is provided under the element layer 20, a sacrificial layer for forming the gap may be removed through the hole 20h.

As shown in FIG. 2, the element portion 20E may include a conductive member 20C. Electric power may be supplied to the conductive member 20C, and the temperature of the element portion 20E may rise. The conductive member 20C is, for example, a heater. In one example, the detection target may be detected when the temperature of the element portion 20E has risen. In another example, the detection target adsorbed to the first member 40 may be detached from the first member 40 by increasing the temperature of the first member 40. For example, initialization is performed. The increase in temperature may remove adsorbed water, for example.

As shown in FIGS. 2 and 3, the sensor 110 may further include a base 50s and a first fixed portion 31F. The first fixed portion 31F is fixed to the base 50s. The element portion 20E is supported by the first fixed portion 31F. A first gap g1 may be provided between the base 50s and the element layer 20. Thereby, dissipating of heat from the element layer 20 is suppressed. For example, the characteristics of the change in electrical resistance are stabilized. The sensor 110 may have, for example, a MEMS (Micro Electro Mechanical Systems) structure.

An insulating layer 50L may be provided between the base 50s and the first fixed portion 31F. The insulating layer 50L may be included in the base 50s.

As shown in FIGS. 1 and 2, the sensor 110 may further include a first connecting portion 31c. The first connecting portion 31c is supported by the first fixed portion 31F. The first connecting portion 31c supports the element layer 20. The first connecting portion 31c may have, for example, a meandering structure. The first connecting portion 31c may have a beam structure. Heat conduction is suppressed. For example, high sensitivity is easily obtained.

As shown in FIGS. 1 and 2, the sensor 110 may further include a second fixed portion 32F. The second fixed portion 32F is fixed to the base 50s. The element portion 20E is further supported by the second fixed portion 32F. The sensor 110 may further include a second connecting portion 32c. The second connecting portion 32c is supported by the second fixed portion 32F. The second connecting portion 32c supports the element layer 20.

For example, a direction from the first fixed portion 31F to the second fixed portion 32F is along a plane including the first direction D1 and the second direction D2. For example, the position of the element portion 20E in the second direction D2 is located between the position of the first fixed portion 31F in the second direction D2 and the position of the second fixed portion 32F in the second direction D2.

As shown in FIG. 1, the sensor 110 may further include a third fixed portion 33F. The third fixed portion 33F is fixed to the base 50s. The element portion 20E is further supported by the third fixed portion 33F. The sensor 110 may further include a third connecting portion 33c. The third connecting portion 33c is supported by the third fixed portion 33F. The third connecting portion 33c supports the element layer 20.

As shown in FIG. 1, the sensor 110 may further include a fourth fixed portion 34F. The fourth fixed portion 34F is fixed to the base 50s. The element portion 20E is further supported by the fourth fixed portion 34F. The sensor 110 may further include a fourth connecting portion 34c. The fourth connecting portion 34c is supported by the fourth fixed portion 34F. The fourth connecting portion 34c supports the element layer 20.

The plurality of connecting portions may have, for example, a meandering structure. The plurality of connecting portions may have a beam structure. Thermal conduction is suppressed. For example, high sensitivity is easily obtained.

It is preferable that the number of the plurality of connecting portions is four or more. The shape of the element layer 20 is stabilized. When the first member 40 is provided on the element layer 20 by a coating method or the like, it is easy to stably form a layer of liquid including the plurality of first particles 41 on the element layer 20. The number of the plurality of connecting portions may be six or more. The number of the plurality of connecting portions may be eight or more. It becomes easy to obtain the element layer 20 having a more stable shape. The first member 40 can be formed more stably.

As shown in FIG. 1, the element portion 20E may include a plurality of first electrodes 11 and a plurality of second electrodes 12. These plurality of electrodes are along the first direction D1. One of the plurality of first electrodes 11 is located between one of the plurality of second electrodes 12 and another one of the plurality of second electrodes 12. One of the plurality of second electrodes 12 is located between one of the plurality of first electrodes 11 and another one of the plurality of first electrodes 11. The first recess 20d overlaps the plurality of first electrodes 11 and the plurality of second electrodes 12.

For example, the first region 21, the second region 22, and the third region 23 may be provided in each of the plurality of first electrodes 11. The first inter-electrode region 21A, the second inter-electrode region 22A, and the third inter-electrode region 23A may be provided between one of the plurality of first electrodes 11 and one of the plurality of second electrodes 12. As shown in FIG. 1, the element portion 20E may include a first connection electrode 11T and a second connection electrode 12T. The first connection electrode 11T electrically connects the plurality of first electrodes 11 to each other. The second connection electrode 12T electrically connects the plurality of second electrodes 12 to each other. For example, the plurality of first electrodes 11 and the plurality of second electrodes 12 are provided between the first connection electrode 11T and the second connection electrode 12T. The plurality of first electrodes 11 and the plurality of second electrodes 12 may form, for example, comb-tooth electrodes.

In one example, the electrical resistance between the first connection electrode 11T and the second connection electrode 12T may be detected. These connection electrodes may function as terminals. In one example, an electrically conducting layer electrically connected to the first electrode 11 may pass through the connecting portion (e.g., the first connecting portion 31c). In one example, an electrically conducting layer electrically connected to the second electrode 12 may pass through a connecting portion (e.g., the second connecting portion 32c).

As shown in FIG. 1, in this example, a plurality of the first recesses 20d are provided. The plurality of first recesses 20d are along the first direction D1. A group including the plurality of first recesses 20d may be aligned along the second direction D2. For example, the plurality of first recesses 20d may be along the first direction D1 and the second direction D2. When the first member 40 is formed by a coating method, the surface energy of the liquid used makes it easy for the first member 40 to be uniformly distributed within the surface according to the plurality of first recesses 20d.

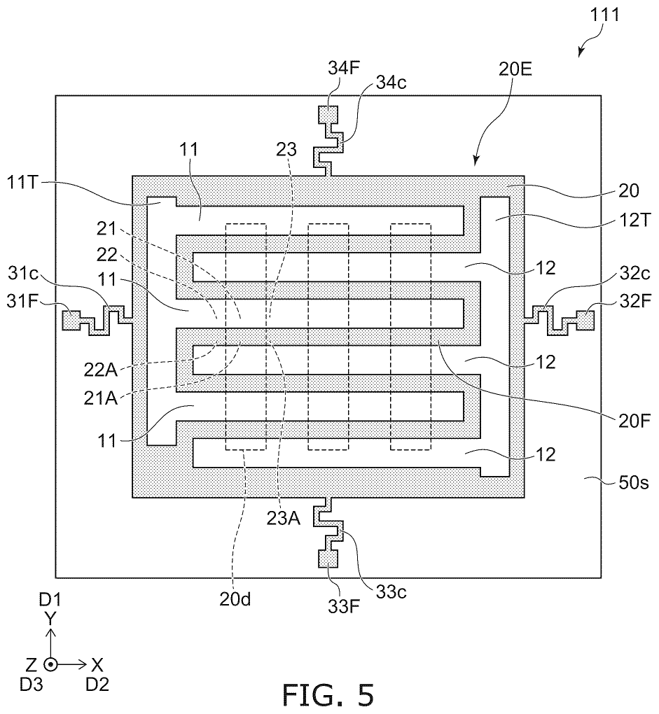

FIG. 5 is a schematic plan view illustrating a sensor according to the first embodiment.

As shown in FIG. 5, in a sensor 111 according to the embodiment, the first face 20F includes the first recess 20d being band-shaped along first direction D1. Except for this, the configuration of the sensor 111 may be the same as the configuration of the sensor 110, for example.

In the sensor 111, the first region 21 is included in the first recess 20d being band-shaped. The first inter-electrode region 21A is included in the first recess 20d being band-shaped. In the sensor 111, the first member 40 (plurality of first particles 41) efficiently gathers in the first recess 20d being band-shaped. High accuracy is stably obtained.

In the sensor 111, the element portion 20E also includes the element layer 20 including the first face 20F, the first electrode 11 provided on the first face 20F, the second electrode 12 provided on the first face 20F, and the first member 40 including the oxide. The direction from the second electrode 12 to the first electrode 11 is along the first direction D1. The first face 20F includes the first inter-electrode region 21A and the second inter-electrode region 22A. The position of the first inter-electrode region 21A in the first direction D1 and the position of the second inter-electrode region 22A in the first direction D1 are located between the position of the second electrode 12 in the first direction D1 and the position of the first electrode 11 in the first direction D1. The direction from the second inter-electrode position of the second inter-electrode region 22A in the second direction D2 to the first inter-electrode position of the first inter-electrode region 21A in the second direction D2 is along the second direction D2. The second direction D2 crosses the first direction D1. The first inter-electrode region 21A is recessed with respect to the second inter-electrode region 22A. At least a part of the first inter-electrode region 21A overlaps the first member 40 in the third direction D3. The third direction D3 crosses a plane including the first direction D1 and the second direction D2.

In the sensor 111, the first member 40 does not overlap the second inter-electrode region 22A in the third direction D3. Alternatively, the first member 40 includes the first inter-electrode portion 40p overlapping the first inter-electrode region 21A in the third direction D3, and a second inter-electrode portion 40q overlapping the second inter-electrode region 22A in the third direction D3. The first inter-electrode amount of the oxide (plurality of first particles 41) per unit area in the first inter-electrode portion 40p is greater than the second inter-electrode amount of the oxide (plurality of first particles 41) per unit area in the second inter-electrode portion 40q. The first member 40 efficiently gathers in the first recess 20d.

In the sensor 111, the first electrode 11 also extends along the second direction D2. The second electrode 12 also extends along the second direction D2.

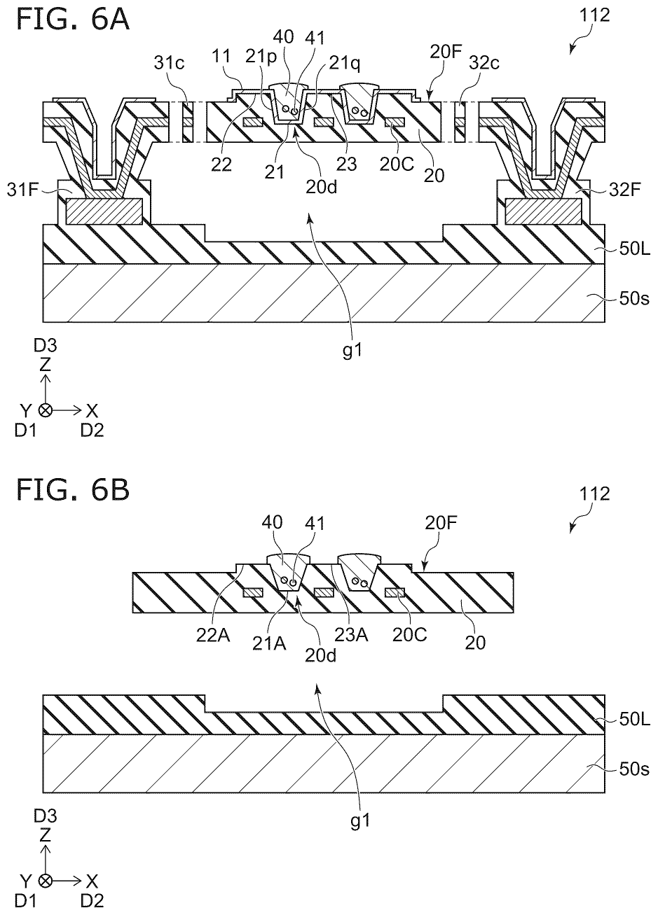

FIGS. 6A and 6B are schematic cross-sectional views illustrating a sensor according to the first embodiment.

FIG. 6A is a cross-sectional view corresponding to the line A1-A2 in FIG. 1. FIG. 6B is a cross-sectional view corresponding to the line A3-A4 in FIG. 1. As shown in FIG. 6A, in a sensor 112 according to the embodiment, the side face of first recess 20d is inclined. Except for this, the configuration of the sensor 112 according to the embodiment may be the same as the configuration of the sensor 110 or the sensor 111, for example.

In the sensor 112, the first face 20F includes the first side face 21p and the second side face 21q. The first side face 21p is located between the first region 21 and the second region 22. The second side face 21q is located between the first region 21 and the third region 23. At least a part of the first side face 21p may be inclined with respect to the third direction D3. At least a part of the second side face 21q may be inclined with respect to the third direction D3. By the inclined side faces, it becomes easier to control the in-plane distribution of the first member 40, for example. For example, by the inclined side faces, it becomes easier for the first member 40 to gather in the first recess 20d, for example.

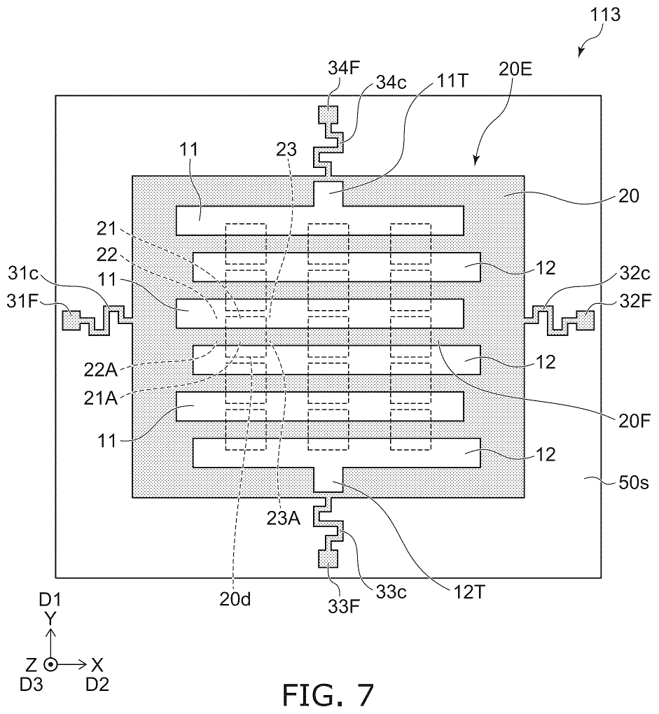

FIG. 7 is a schematic plan view illustrating a sensor according to the first embodiment.

As shown in FIG. 7, in a sensor 113 according to the embodiment, the shapes of the first electrode 11 and the second electrode 12 differ from those in the sensor 110. Except for this, the configuration of sensor 113 may be the same as the configuration of sensor 110, etc.

In the sensor 113, the first connection electrode 11T is electrically connected to one of the plurality of first electrodes 11. The second connection electrode 12T is electrically connected to one of the plurality of second electrodes 12. The plurality of second electrodes 12 may be considered to be part of the plurality of first electrodes 11. In this case, one of the plurality of first electrodes 11 may be considered to be the “first electrode 11” and another one of the plurality of first electrodes 11 may be considered to be the “second electrode 12.”

The sensor 113 also has the first recess 20d. For example, the first member 40 efficiently gathers in the first recess 20d. The sensor 113 also has stable characteristics.

In the sensor 113, at least a part of the first recess 20d is located between the first electrode 11 and the second electrode 12. The electrical resistance between the first electrode 11 and the second electrode 12 changes depending on a change in the characteristics of the first member 40 provided in at least a part of the first recess 20d. The change in the characteristics of the first member 40 depends on the detection target.

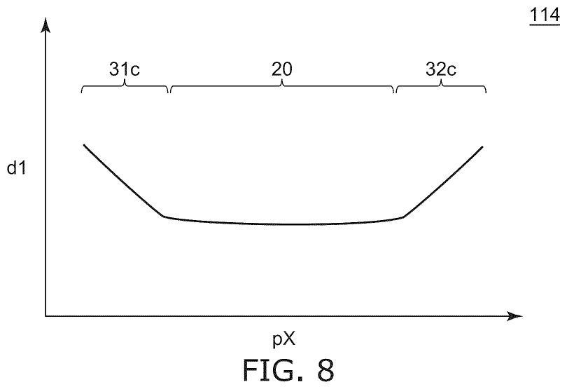

FIG. 8 is a graph illustrating a sensor according to the first embodiment.

FIG. 8 illustrates the characteristics of a sensor 114 according to the embodiment. The configuration of the sensor 114 may have any of the configurations of the sensors described above. The horizontal axis of FIG. 8 is the position px in the X-axis direction. The vertical axis is the distance d1 (see FIG. 1). In the region corresponding to the element layer 20, the distance d1 is the distance d1 along the third direction D3 between the base 50s and the element layer 20. In the region corresponding to the first connecting portion 31c, the distance d1 is the distance along the third direction D3 between the base 50s and the first connecting portion 31c. In the region corresponding to the second connecting portion 32c, the distance d1 is the distance along the third direction D3 between the base 50s and the second connecting portion 32c.

As shown in FIG. 8, the distance d1 in the region corresponding to the element layer 20 may be shorter than the distance d1 in the region corresponding to the first connecting portion 31c. The distance d1 in the region corresponding to the element layer 20 may be shorter than the distance d1 in the region corresponding to the second connecting portion 32c. For example, the first member 40 can be easily and stably formed on the element layer 20.

As shown in FIG. 8, in the region corresponding to the element layer 20, the distance d1 may change to a downward convex shape. For example, the element layer 20 may be convex toward the base 50s. Thereby, the first member 40 can be stably formed on the element layer 20, for example. For example, when the first member 40 is formed by a coating method, a film including a liquid that includes the plurality of first particles 41 is easier to be stably formed on the element layer 20.

### Second Embodiment

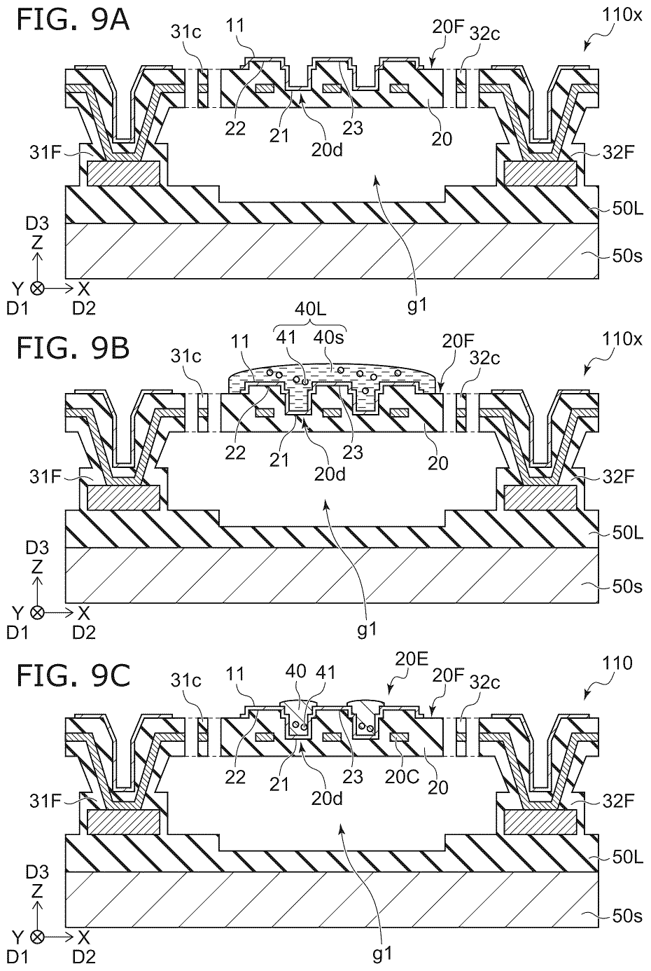

FIGS. 9A to 9C are schematic cross-sectional views illustrating a method for manufacturing a sensor according to the second embodiment.

As shown in FIG. 9A, a structure 110x is prepared. The structure 110x includes the element layer 20 and the first electrode 11. The element layer 20 includes the first face 20F including the first recess 20d. The first electrode 11 is provided on the first face 20F. The structure 110x may include the plurality of first electrodes 11. One of the plurality of first electrodes 11 may be regarded as the second electrode 12 (see FIG. 1). The structure 110x may include the base 50s, the fixed portion (e.g., the first fixed portion 31F, etc.), and the connecting portion (e.g., the first connecting portion 31c, etc.). The first gap g1 may be provided between the base 50s and the element layer 20. The structure 110x may include a MEMS structure.

As shown in FIG. 9B, a liquid film 40L is formed in the first recess 20d of the structure 110x. The liquid film 40L includes an oxide (e.g., the plurality of first particles 41) and a solvent 40s. The solvent 40s may be, for example, an organic solvent. The organic solvent may include, for example, ethylene glycol.

As shown in FIG. 9C, at least a part of the solvent 40s is removed to form at least a part of the first member 40 including the oxide (the plurality of first particles 41) in the first recess 20d. Thereby, the sensor 110 can be obtained, for example. According to the embodiment, a method for manufacturing a sensor capable of obtaining stable characteristics can be provided.

The method for manufacturing according to the embodiment may include forming the first recess 20d. For example, the first recess 20d can be formed by removing a part of the element layer 20. The removing the part of the element layer 20 may include, for example, etching. The etching may include at least one of wet etching or dry etching.

The embodiment may include the following Technical proposals:

### (Technical Proposal 1)

A sensor, comprising:
an element portion,
the element portion includes:
   an element layer including a first face;
   a first electrode provided on the first face;
   a second electrode provided on the first face, a direction from the second electrode to the first electrode being along a first direction; and
   a first member including an oxide,
the first face including a first region and a second region,
a direction from a second position in a second direction of the second region to a first position in the second direction of the first region being along the second direction,
the second direction crossing the first direction,
the first region being recessed with respect to the second region,
the first region and the second region overlapping at least a part of the first electrode in a third direction,
the third direction crossing a plane including the first direction and the second direction, and
at least a part of the first electrode being between the first region and the first member in the third direction.

### (Technical Proposal 2)

The sensor according to Technical proposal 1, wherein the first electrode extends along the second direction, and the second electrode extends along the second direction.

### (Technical Proposal 3)

The sensor according to Technical proposal 1 or 2, wherein
the first electrode and the second electrode are configured such that an electrical resistance between the first electrode and
the second electrode changes depending on a detection target around the element portion.

### (Technical Proposal 4)

The sensor according to any one of Technical proposals 1-3, wherein
the first member does not overlap the second region in the third direction, or the first member includes a first overlapping portion overlapping the first region in the third direction and a second overlapping portion overlapping the second region in the third direction, and
a first amount of the oxide per unit area in the first overlapping portion is greater than a second amount of the oxide per unit area in the second overlapping portion.

### (Technical Proposal 5)

The sensor according to any one of Technical proposals 1-4, wherein
the first face further includes a third region,
a first position of the first region in the second direction is between a second position of the second region in the second direction and a third position of the third region in the second direction,
the first region is recessed with respect to the third region, and
the third region overlaps a part of the first electrode in the third direction.

### (Technical Proposal 6)

The sensor according to Technical proposal 5, wherein
the first face further includes a first side face between the first region and the second region, and a second side face between the first region and the third region,
the first side face and the second side face cross the second direction,
a part of the first electrode is between the first side face and at least a part of the first member, and
another part of the first electrode is between the at least the part of the first member and the second side face.

### (Technical Proposal 7)

The sensor according to Technical proposal 6, wherein
at least a part of the first side face is inclined with respect to the third direction.

### (Technical Proposal 8)

The sensor according to any one of Technical proposals 1-7, wherein
the first face further includes a first inter-electrode region,
a position of the first inter-electrode region in the first direction is between a position of the second electrode in the first direction and a position of the first electrode in the first direction,
a direction from the position of the first inter-electrode region in the first direction to a position of the first region in the first direction is along the first direction,
the first inter-electrode region is recessed with respect to the second region, and
at least a part of the first inter-electrode region overlaps the first member in the third direction.

### (Technical Proposal 9)

The sensor according to any one of Technical proposals 1-3, wherein
the first face further includes a first inter-electrode region and a second inter-electrode region,
a position of the first inter-electrode region in the first direction and a position of the second inter-electrode region in the first direction are between a position of the second electrode in the first direction and a position of the first electrode in the first direction,
a direction from a second inter-electrode position in the first direction of the second inter-electrode region to a first inter-electrode position in the first direction of the first inter-electrode region is along the second direction,
the first inter-electrode region is recessed with respect to the second inter-electrode region, and
at least a part of the first inter-electrode region overlaps the first member in the third direction.

### (Technical Proposal 10)

The sensor according to Technical proposal 9, wherein
the first member does not overlap the second inter-electrode region in the third direction, or the first member includes a first inter-electrode portion overlapping the first inter-electrode region in the third direction and a second inter-electrode portion overlapping the second inter-electrode region in the third direction,
a first inter-electrode amount of the oxide per unit area in the first inter-electrode portion is greater than a second inter-electrode amount of the oxide per unit area in the second inter-electrode portion.

### (Technical Proposal 11)

The sensor according to any one of Technical proposals 1-10, wherein
the first face includes a first recess being band-shaped along the first direction, and
the first region is included in the first recess.

### (Technical Proposal 12)

A sensor, comprising:
an element portion,
the element portion includes:
   an element layer including a first face;
   a first electrode provided on the first face;
   a second electrode provided on the first face, a direction from the second electrode to the first electrode being along a first direction; and
   a first member including an oxide,
the first face including a first inter-electrode region and a second inter-electrode region,
a position of the first inter-electrode region in the first direction and a position of the second inter-electrode region in the first direction being between a position of the second electrode in the first direction and a position of the first electrode in the first direction,
a direction from a second inter-electrode position in a second direction of the second inter-electrode region to a first inter-electrode position in the second direction of the first inter-electrode region being along a second direction,
the second direction crossing the first direction,
the first inter-electrode region being recessed with respect to the second inter-electrode region,
at least a part of the first inter-electrode region overlapping the first member in a third direction, the third direction crossing a plane including the first direction and the second direction.

### (Technical Proposal 13)

The sensor according to Technical proposal 12, wherein
the first member does not overlap the second inter-electrode region in the third direction, or the first member includes a first inter-electrode portion overlapping the first inter-electrode region in the third direction and a second inter-electrode portion overlapping the second inter-electrode region in the third direction, and
a first inter-electrode amount of the oxide per unit area in the first inter-electrode portion is greater than a second inter-electrode amount of the oxide per unit area in the second inter-electrode portion.

### (Technical Proposal 14)

The sensor according to Technical proposal 12 or 13, wherein
the first electrode extends along the second direction, and
the second electrode extends along the second direction.

### (Technical Proposal 15)

The sensor according to any one of Technical proposals 12-14, wherein
the first face includes a first recess being band-shaped along the first direction, and
the first inter-electrode region is included in the first recess.

### (Technical Proposal 16)

The sensor according to Technical proposal 11 or 15, wherein
the element portion includes a plurality of the first electrodes and a plurality of the second electrodes,
one of the plurality of first electrodes is between one of the plurality of second electrodes and another one of the plurality of second electrodes,
the one of the plurality of second electrodes is between the one of the plurality of first electrodes and another one of the plurality of first electrodes, and
the first recess overlaps the plurality of first electrodes and the plurality of second electrodes.

### (Technical Proposal 17)

The sensor according to any one of Technical proposals 1-16, further comprising:
a base; and
a first fixed portion fixed to the base,
the element portion being supported by the first fixed portion, and
a first gap being provided between the base and the element layer.

### (Technical Proposal 18)

The sensor according to Technical proposal 17, wherein
the element layer is convex toward the base.

### (Technical Proposal 19)

The sensor according to any one of Technical proposals 1-18, wherein
the first member includes a plurality of first particles including an oxide,
the oxide includes oxygen and at least one selected from the group consisting of tin, zinc, tungsten, molybdenum, and indium, and
at least one of the first electrode or the second electrode includes at least one selected from the group consisting of platinum, gold, silver, copper, aluminum, and titanium nitride.

### (Technical Proposal 20)

A method for manufacturing a sensor, the method comprising:
forming a liquid film in a first recess of a structure including an element layer, the liquid film including an oxide and a solvent, the element layer including a first face including the first recess and a first electrode provided on the first face; and
removing at least a part of the solvent to form at least a part of a first member including the oxide in the first recess.

According to the embodiment, a sensor capable of obtaining stable characteristics and a method for manufacturing the same are provided.

In the specification, “electrically connected” includes a state in which plurality of conductors are physically in contact with each other and current flows between these plurality of conductors. “Electrically connected” includes a state in which a conductor is inserted between plurality of conductors and current flows between these plurality of conductors.

Hereinabove, exemplary embodiments of the invention are described with reference to specific examples. However, the embodiments of the invention are not limited to these specific examples. For example, one skilled in the art may similarly practice the invention by appropriately selecting specific configurations of components included in sensors such as element layers, electrodes, first members, bases, fixed portions, connecting portions, etc., from known art. Such practice is included in the scope of the invention to the extent that similar effects thereto are obtained.

Further, any two or more components of the specific examples may be combined within the extent of technical feasibility and are included in the scope of the invention to the extent that the purport of the invention is included.

Moreover, all sensors and all method for manufacturing the same practicable by an appropriate design modification by one skilled in the art based on the sensors and the method for manufacturing the same described above as embodiments of the invention also are within the scope of the invention to the extent that the purport of the invention is included.

Various other variations and modifications can be conceived by those skilled in the art within the spirit of the invention, and it is understood that such variations and modifications are also encompassed within the scope of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A sensor, comprising:
an element portion,
the element portion includes:
an element layer including a first face;
a first electrode provided on the first face;
a second electrode provided on the first face, a direction from the second electrode to the first electrode being along a first direction; and
a first member including an oxide,
the first face including a first region and a second region,
a direction from a second position in a second direction of the second region to a first position in the second direction of the first region being along the second direction,
the second direction crossing the first direction,
the first region being recessed with respect to the second region,
the first region and the second region overlapping at least a part of the first electrode in a third direction,
the third direction crossing a plane including the first direction and the second direction, and
at least a part of the first electrode being between the first region and the first member in the third direction.

2. The sensor according to claim 1, wherein
the first electrode extends along the second direction, and
the second electrode extends along the second direction.

3. The sensor according to claim 1, wherein
the first electrode and the second electrode are configured such that an electrical resistance between the first electrode and the second electrode changes depending on a detection target around the element portion.

4. The sensor according to claim 1, wherein
the first member does not overlap the second region in the third direction, or the first member includes a first overlapping portion overlapping the first region in the third direction and a second overlapping portion overlapping the second region in the third direction, and
a first amount of the oxide per unit area in the first overlapping portion is greater than a second amount of the oxide per unit area in the second overlapping portion.

5. The sensor according to claim 1, wherein
the first face further includes a third region,
a first position of the first region in the second direction is between a second position of the second region in the second direction and a third position of the third region in the second direction,
the first region is recessed with respect to the third region, and
the third region overlaps a part of the first electrode in the third direction.

6. The sensor according to claim 5, wherein
the first face further includes a first side face between the first region and the second region, and a second side face between the first region and the third region,
the first side face and the second side face cross the second direction,
a part of the first electrode is between the first side face and at least a part of the first member, and
another part of the first electrode is between the at least the part of the first member and the second side face.

7. The sensor according to claim 6, wherein
at least a part of the first side face is inclined with respect to the third direction.

8. The sensor according to claim 1, wherein
the first face further includes a first inter-electrode region,
a position of the first inter-electrode region in the first direction is between a position of the second electrode in the first direction and a position of the first electrode in the first direction,
a direction from the position of the first inter-electrode region in the first direction to a position of the first region in the first direction is along the first direction,
the first inter-electrode region is recessed with respect to the second region, and
at least a part of the first inter-electrode region overlaps the first member in the third direction.

9. The sensor according to claim 1, wherein
the first face further includes a first inter-electrode region and a second inter-electrode region,
a position of the first inter-electrode region in the first direction and a position of the second inter-electrode region in the first direction are between a position of the second electrode in the first direction and a position of the first electrode in the first direction,
a direction from a second inter-electrode position in the first direction of the second inter-electrode region to a first inter-electrode position in the first direction of the first inter-electrode region is along the second direction,
the first inter-electrode region is recessed with respect to the second inter-electrode region, and
at least a part of the first inter-electrode region overlaps the first member in the third direction.

10. The sensor according to claim 9, wherein
the first member does not overlap the second inter-electrode region in the third direction, or the first member includes a first inter-electrode portion overlapping the first inter-electrode region in the third direction and a second inter-electrode portion overlapping the second inter-electrode region in the third direction,
a first inter-electrode amount of the oxide per unit area in the first inter-electrode portion is greater than a second inter-electrode amount of the oxide per unit area in the second inter-electrode portion.

11. The sensor according to claim 1, wherein
the first face includes a first recess being band-shaped along the first direction, and
the first region is included in the first recess.

12. A sensor, comprising:
an element portion,
the element portion includes:
an element layer including a first face;
a first electrode provided on the first face;
a second electrode provided on the first face, a direction from the second electrode to the first electrode being along a first direction; and
a first member including an oxide,
the first face including a first inter-electrode region and a second inter-electrode region,
a position of the first inter-electrode region in the first direction and a position of the second inter-electrode region in the first direction being between a position of the second electrode in the first direction and a position of the first electrode in the first direction,
a direction from a second inter-electrode position in a second direction of the second inter-electrode region to a first inter-electrode position in the second direction of the first inter-electrode region being along a second direction,
the second direction crossing the first direction,
the first inter-electrode region being recessed with respect to the second inter-electrode region,
at least a part of the first inter-electrode region overlapping the first member in a third direction,
the third direction crossing a plane including the first direction and the second direction.

13. The sensor according to claim 12, wherein
the first member does not overlap the second inter-electrode region in the third direction, or the first member includes a first inter-electrode portion overlapping the first inter-electrode region in the third direction and a second inter-electrode portion overlapping the second inter-electrode region in the third direction, and
a first inter-electrode amount of the oxide per unit area in the first inter-electrode portion is greater than a second inter-electrode amount of the oxide per unit area in the second inter-electrode portion.

14. The sensor according to claim 12, wherein
the first electrode extends along the second direction, and
the second electrode extends along the second direction.

15. The sensor according to claim 12, wherein
the first face includes a first recess being band-shaped along the first direction, and
the first inter-electrode region is included in the first recess.

16. The sensor according to claim 11, wherein
the element portion includes a plurality of the first electrodes and a plurality of the second electrodes,
one of the plurality of first electrodes is between one of the plurality of second electrodes and another one of the plurality of second electrodes,
the one of the plurality of second electrodes is between the one of the plurality of first electrodes and another one of the plurality of first electrodes, and
the first recess overlaps the plurality of first electrodes and the plurality of second electrodes.

17. The sensor according to claim 1, further comprising:
a base; and
a first fixed portion fixed to the base,
the element portion being supported by the first fixed portion, and
a first gap being provided between the base and the element layer.

18. The sensor according to claim 17, wherein
the element layer is convex toward the base.

19. The sensor according to claim 1, wherein
the first member includes a plurality of first particles including an oxide,
the oxide includes oxygen and at least one selected from the group consisting of tin, zinc, tungsten, molybdenum, and indium, and
at least one of the first electrode or the second electrode includes at least one selected from the group consisting of platinum, gold, silver, copper, aluminum, and titanium nitride.

20. A method for manufacturing a sensor, the method comprising:
forming a liquid film in a first recess of a structure including an element layer, the liquid film including an oxide and a solvent, the element layer including a first face including the first recess and a first electrode provided on the first face; and
removing at least a part of the solvent to form at least a part of a first member including the oxide in the first recess.
